(19) United States
(12) Patent Application Publication
(10) Pub. No.: US 2009/0024063 A1
(43) Pub. Date: Jan. 22, 2009

(54) Penile Light and Pump Device and Method

(76) Inventor: Veerachat Kalvatanond, Nonthaburi (TH)

Correspondence Address: John J. Connors;Connors & Associates, Inc., 1600 Dove Street #220, Newport Beach, CA, 92660, US

(21) Appl. No.: 12/087,714

(22) PCT Filed: Jan. 11, 2006
(86) PCT No.: PCT/US2006/001166

§ 371 Date: Jul. 10, 2008

(51) Int. Cl. A61B 18/18; A61H 1/00; A61F 5/00

(52) U.S. Cl. 601/15; 606/3; 606/10; 600/38

(57) ABSTRACT

Penile illuminating equipment includes a plurality of light emitting diodes positioned on a mounting structure in a manner to illuminate a penis with light emitted from the diodes along the penis' length and circumference. Optionally, a penis enlargement device may be used in combination with the penile illuminating equipment.

### INCORPORATION BY REFERENCE

The inventor incorporates herein by reference any and all U.S. patents, U.S. patent applications, and other documents, hard copy or electronic, cited or referred to in this application.

### DEFINITIONS

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

### BACKGROUND OF INVENTION

NASA and others have developed LED (light emitting diode) therapy to accelerate healing, to reduce pain, to treat muscles, to provide acupoint stimulation, and other applications. In accordance with this invention, LED therapy is used to improve the health of a penis. Moreover, LED therapy may be combined with a treatment to increase the size of a penis.

### SUMMARY OF INVENTION

This invention has one or more features as discussed subsequently herein. After reading the following section entitled “DETAILED DESCRIPTION OF ONE EMBODIMENT OF THIS INVENTION,” one will understand how the features of this invention provide its benefits. The benefits of this invention include, but are not limited to: improved health of the penis, increasing the size of the penis, and improved blood circulation in the penis.

Without limiting the scope of this invention as expressed by the claims that follow, some, but not necessarily all, of its features are:

One, the penile illuminating equipment of this invention includes a plurality of light emitting diodes positioned on a mounting structure in a manner to illuminate a penis with light emitted from the diodes. The diodes may be positioned to illuminate the penis substantially along the entire length and circumference of the penis. The light from the diodes is essentially monochromatic light having a wavelength in the range substantially from 400 to 990 nanometers. An array of diodes may be use that all emit light of the same wavelength or the array may comprises a plurality of diodes with individual diodes in the array emitting light of different wavelengths.

Two, the diodes constantly turn on and off, and the penile illuminating equipment includes a control unit for regulating the frequency of the diodes, maintaining the frequency in a range substantially from 0.1 Hertz to 60 Kilo-Hertz. Each diode is maintained at the same frequency, but the control unit may periodically switch essentially all the diodes from one constant frequency to another constant frequency. The control unit may automatically discontinue illumination after a predetermined treatment period, for example, substantially 15 minutes.

Three, the diode frequency may be modulated in different ways. For example, it may be modulated by starting at a first substantially constant predetermined frequency for a predetermined period, then switching to a second substantially constant predetermined frequency for a predetermined period, and lastly switching to a third substantially constant predetermined frequency for a predetermined period. The predetermined period may be the same or different and may be no greater than substantially 5 minutes. In one embodiment, the first frequency is at a relatively low, minimum first frequency, then is switched to a second frequency that is at a maximum high frequency, and finally is switched to a third frequency that is intermediate the first and second frequencies. For example, the first frequency is approximately 44 Hertz, the second frequency is approximately 292 Hertz, and the third frequency is approximately 146 Hertz.

Four, the penile illuminating equipment of this invention may comprise a housing forming a chamber sized to receive a penis to be illuminated. The chamber may have an opening sized to enable the penis to be illuminated to be inserted into the chamber through the opening. The light emitting diodes are positioned on the chamber to illuminate the penis with light from the diodes. A control unit regulates the frequency of the light from the diodes. The housing may be an elongated tubular structure having a longitudinal centerline and an open end to receive a penis to be illuminated. The penis extends lengthwise in the chamber substantially along the longitudinal centerline. An elongated support member may extend longitudinally from the open end along the chamber to form a portion of a side of the housing. The light emitting diodes may be mounted on the support member in a linear array, with individual diodes being spaced apart and each diode oriented to direct light inward towards the longitudinal centerline.

Five, this invention may comprise the combination of the penile illuminating equipment of this invention and a penile enlargement device. The penile enlargement device may be a pumping apparatus. This pumping apparatus may be a hollow tubular structure open at one end and closed at the other. The penis is inserted into the open end, which is pressed firmly against the patient's body at the root of the penis. This at least partially seals and closes the open end. An elastic, resilient bulb at the closed end that, upon manually squeezing, pumps air from the tubular structure, at least partially evacuating the tubular structure, causing enlargement of the penis. The enlarged penis is illuminated with light from the diodes.

These features are not listed in any rank order nor is this list intended to be exhaustive.

This invention also includes a method of enhancing the health of a penis. This method comprises the step of illuminating the penis substantially along the penis' entire length and circumference with light from a plurality of light emitting diodes. The frequency of the light may be modulated by switching from one essentially constant frequency to another essentially constant frequency. The penis may be enlarged using the pumping apparatus as discussed above and either concurrently or sequentially illuminated with light from the diodes as discussed above. The illumination and enlargement may be for a period substantially from 5 to 15 minutes for no more than substantially 5 times per day.

### DESCRIPTION OF THE DRAWING

One embodiment of this invention, illustrating all its features, will now be discussed in detail. This embodiment depicts the novel and non-obvious penile light and pump device and method of this invention as shown in the accompanying drawing, which is for illustrative purposes only. This drawing includes the following figures (Figs.), with like numerals indicating like parts:

FIG. 1 is a side view of the penile light and pump device of this invention showing a conventional penile pumping apparatus aligned to be inserted into one open end of the penile illuminating equipment of this invention.

FIG. 2 is a perspective view showing the penile illuminating equipment of this invention.

FIG. 3 is a schematic diagram of an electrical circuit for the control unit shown in FIG. 2 for the penile illuminating equipment of this invention.

FIG. 4 is a process flow diagram illustrating the program for the central processing unit employed in the electrical circuit depicted in FIG. 3.

### DETAILED DESCRIPTION OF ONE EMBODIMENT OF THIS INVENTION

### General

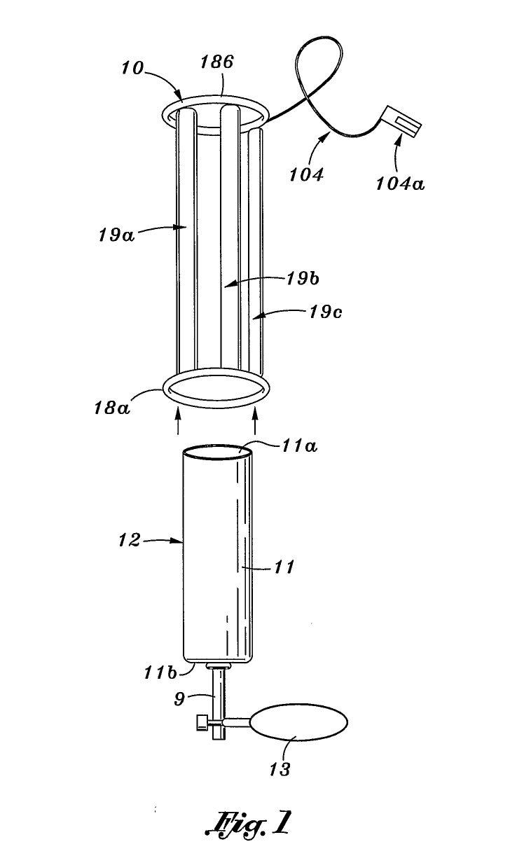

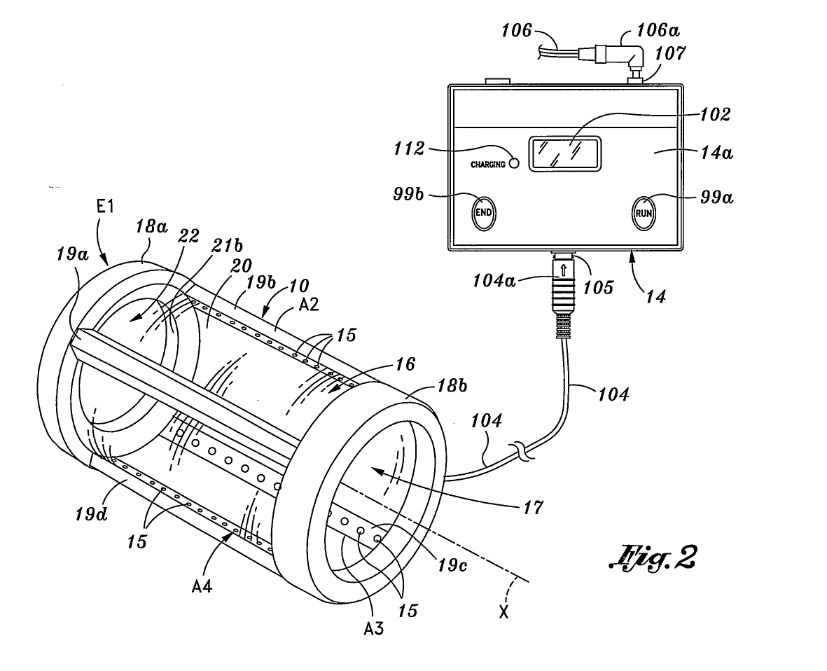

This invention comprises the penile illuminating equipment 10 standing alone as shown FIG. 2, or as shown in FIG. 1, in combination with a convention penile pumping apparatus 12. Such a penile pumping apparatus 12 may be purchased from Fastsize. LLC, of Irvine, Calif. The pumping apparatus 12 includes a hollow, cylindrical, transparent or translucent tube 11 open at its one end 11a and closed at its other end 11b. An elastic, resilient bulb 13 at the closed end 11b is attached to a hollow stem 9 that is in communication with the interior of the tube 11. The dimensions of the tube 11 are such that the tube 11 fits within and is essentially surrounded by a chamber 17 (FIG. 2) of the penile illuminating equipment 10 upon inserting the pumping apparatus 12 therein.

The penile illuminating equipment 10 illuminates a penis (not shown) inserted lengthwise into the equipment with light from diodes 15. The diode light illuminates the penis substantially along its entire length and circumference. The diodes 15 turn on and off rapidly at a frequency that is controlled in accordance with this invention. Typically, they are maintained for a fixed period at one frequency and switched to another frequency for a fixed period. This frequency may be from about 0.1 Hertz to about 60 Kilo-Hertz. The period the diodes 15 are maintained at any given frequency may typically be about 5 minutes. The equipment 10 includes a control unit 14 for regulating the frequency of the light from the diodes 15 as discussed in greater detail subsequently. The light from the diodes 15 is essentially monochromatic light having a wavelength from 400 to 990 nanometers.

When the pumping apparatus 12 is used in combination with penile illuminating equipment 10, the penis is inserted into the chamber 17 and then the pumping apparatus 12 is inserted into this chamber. The patient advances the pumping apparatus 12 inward to enclose the penis within the tube 11, pressing the open end 11a firmly against the patient's body at the root of the penis. This at least partially seals and closes the open end 11a. The patient manually squeezes the bulb 13, pumping air from the tube 11 to at least partially evacuate the tube, causing enlargement of the penis. The enlarged penis is illuminated with light from the diodes 15.

### Penile Illuminating Equipment

As best depicted in FIG. 2, the penile illuminating equipment 10 includes an elongated housing 16 forming a chamber 17 sized to receive a penis to be illuminated. The housing 16 may comprise a pair of opposed annular mounting members 18a and 18b connected together by four linear arms 19a, 19b, 19c, and 19d. These arms 19a, 19b, 19c, and 19d are disposed along an outside surface of an optional transparent hollow plastic cylinder 20. The arms 19a, 19b, 19c, and 19d are 90° apart from each other and are substantially parallel to a longitudinal centerline X of the equipment 10. This arrangement forms an open tubular housing 16 having an open end E1 to receive the penis to be illuminated. Other housing structures may be employed so long as they provide a chamber (open or partially closed) that can accommodate a penis to be illuminated.

Each arm 19a, 19b, 19c, and 19d provides an elongated support member extending longitudinally from the end E1 along the chamber 17 to form a portion of a side of the housing 16. Each arm 19a, 19b, 19c, and 19d carries a linear array A1, A2, A3, and A4, respectively, of diodes 15 equally spaced apart from each other and aligned in a row. Each diode 15 is oriented to direct light therefrom inward towards the centerline X thereby illuminating the penis when inserted into the chamber 17. The annular mounting member 18a has an opening 22 that provides an entryway for the penis. The pumping apparatus 12 extends into the chamber 17 through this opening 22 lengthwise substantially along the centerline X.

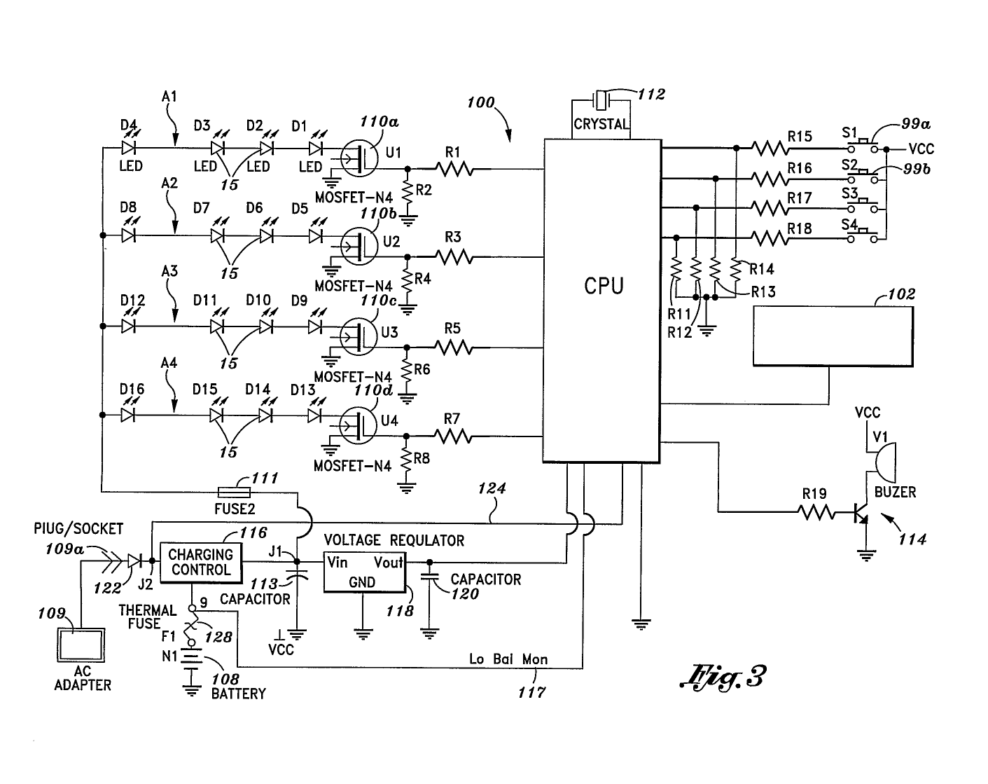

The control unit 14 regulates the frequency of the light from the diodes 15. As illustrated in FIG. 2, it includes a case 14a holding a control circuit 100 shown in FIG. 3. Control buttons 99a and 99b are used to turn the equipment 10 on and off. The button 99a, upon being manually depressed, turns on the diodes 15 and button 99b, upon being manually depressed, turns off the diodes 15. When the button 99a is actuated to turn on the equipment 10, a liquid crystal display 102 shows the frequency the diodes. The diodes 15 are electrically coupled to the circuit 100 through a detachable cable 104 having a terminal end 104a that is inserted into a socket 105 in the case 14a to connect to the circuit 100. Another cable 106 connects the circuit 100 to a source of electrical power. This cable 106 has a terminal end 106a that is detachably connected to another socket 107 in the case 14a. As shown in FIG. 8, electrical energy is provided by a battery 108 or AC current supplied through an AC adapter 109 that converts AC current to DC current. An indicator light 112 on the case 14a when lit indicates that the battery 108 is being charged.

The circuit 100 is designed to discontinue automatically illumination by the diodes 15 after a predetermined time period, typically after about 15 minutes. During this predetermined time period the circuit 100 automatically switches from one essentially constant frequency to another essentially constant frequency.

Referring to FIG. 3, the circuit 100 includes a central processing unit CPU to which the arrays A1, A2, A3, and A4 are electrically connected. The arrays A1, A2, A3, and A4 are in parallel connection, each having one side connected to the CPU through Mosfet transistors 110a, 110b, 110c, and 110d, respectively, and a resistor network including the resistors R1, R2, R3, R4, R5, R6, R7, and R8. The Mosfet transistor 110a is connected to the CPU via the resistor R1, with the resistor R2 having one side grounded and its other side connected between the Mosfet transistors 110a and the resistor R1. The other Mosfet transistors 110b through 110d are connected to the CPU in a similar manner through the pairs of resistors R3 and R4, R5 and R6, and R7, and R8. The other sides of the arrays A1, A2, A3, and A4 are connected to ground via a fuse 111 and capacitor 113. The line between the fuse 111 and the capacitor 113 has a junction J1 that also connects this side of the arrays A1, A2, A3, and A4 between the output of a charging control unit 116 and an input of a grounded voltage regulator 118.

The output of the voltage regulator 118 is connected to the CPU via a grounded capacitor 120. The input to the charging control unit 116 is connected to the output of the AC adapter 109 through a plug socket 109a and a diode 122. A line 124 extends between the CPU and a junction J2 between the diode 122 and the input of the charging control unit 116. The CPU is also connected to the buttons 99a, 99b through a resistor network comprising the resistors R11, R12, R13, R14, R15, R16, R17, and R18. Additional control buttons S3 and S4 may be employed to activate optional features. Electrical energy is provided to the CPU through the voltage regulator 118 and the charging control unit 116, which has an input connected to the battery 108 through a fuse 128. An audio alarm 114 is also connected through a resistor R19 to the CPU and is energized when the battery 108 is low. A line 117 has one end connected between the battery 108 and a voltage regulator 118 and its other end connected to the CPU to provide a signal to the CPU to indicate when the battery is low.

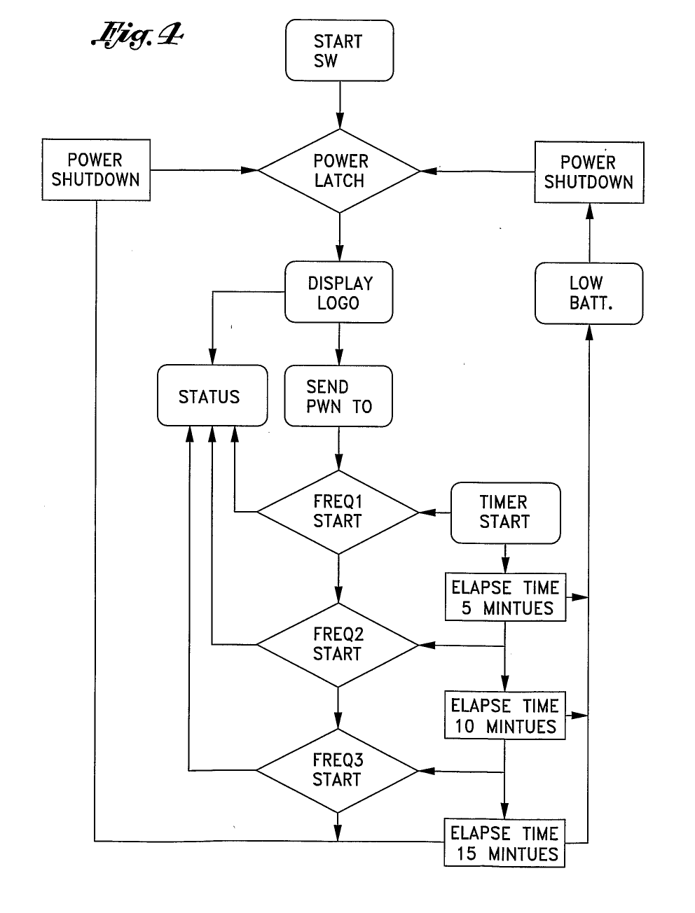

The CPU is programmed in accordance with conventional programming techniques to provide the control functions for the penile illuminating equipment 10, including controlling the frequency of this light from the diodes 15, and the duration of illumination at different frequencies, and the entire period of illumination. Referring to FIG. 4, when the start button 99a is actuated, power is provided to the circuit 100 energizing the CPU. A trademark or logo is briefly displayed on the liquid crystal display 102 and then the CPU operates the diodes 15 at the first frequency and proves a numerical readout of this first frequency that is displayed on the liquid crystal display 102. An internal timer in the CPU also starts to run and at the end of five minutes, the CPU switches the operation of the diodes 15 to the second frequency. The CPU operates the diodes 15 at this second frequency and again proves a numerical readout of this second frequency that is displayed on the liquid crystal display 102. At the end on the second five-minute interval, the CPU switches the operation of the diodes 15 to the third frequency, again providing numerical readout of this third frequency that is displayed on the liquid crystal display 102. At the end of this third five-minute interval, the CPU turns off the penile illuminating equipment 10. In one embodiment of this invention, these frequencies fluctuate over the treatment period and may start at a relatively low, minimum frequency, then increase to a maximum high frequency, and finally drop to an intermediate frequency. For example, the first frequency may be approximately 44 Hertz, the second frequency may be approximately 292 Hertz, and the third frequency may be approximately 146 Hertz. The CPU will also energize the light 112 to indicate that the battery 108 is low and is being charged.

### SCOPE OF THE INVENTION

The above presents a description of the best mode contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiment disclosed. On the contrary, the intention is to cover all modifications and alternate constructions coming within the spirit and scope of the invention as generally expressed by the following claims, which particularly point out and distinctly claim the subject matter of the invention:

1. Penile illuminating equipment including a plurality of light emitting diodes positioned on a mounting structure in a manner to illuminate a penis with light emitted from the diodes.

2. The equipment of claim 1 where the diodes are positioned to illuminate the penis substantially along the entire length and circumference of the penis.

3. The equipment of claim 1 including a control unit for regulating frequency of the light from the diodes so that said light is essentially monochromatic light having a wavelength substantially from 400 to 990 nanometers and a frequency substantially from 0.1 Hertz to 60 Kilo-Hertz.

4. The equipment of claim 3 where the control unit controls the frequency at
a first substantially constant predetermined frequency for a predetermined period,
a second substantially constant predetermined frequency for a predetermined period, and
a third substantially constant predetermined frequency for a predetermined period.

5. The equipment of claim 4 where the first frequency is at a relatively low, minimum frequency, the second frequency is at a maximum high frequency, and the third frequency is intermediate the first and second frequencies.

6. The equipment of claim 5 where the first frequency is substantially 44 Hertz, the second frequency is substantially 292 Hertz, and the third frequency is substantially 146 Hertz.

7. The equipment of claim 4 where the predetermined period is no greater than substantially 5 minutes.

8. The equipment of claim 3 where the control unit discontinues illumination after a predetermined time period.

9. The equipment of claim 1 including an array of diodes wherein substantially all the diodes emit light of substantially the same wavelength.

10. Penile illuminating equipment comprising
an elongated housing forming a chamber sized to receive a penis to be illuminated, said chamber having an opening sized to enable the penis to be illuminated to be inserted into the chamber through the opening,
a plurality of light emitting diodes positioned on the chamber to illuminate the penis with light from the diodes, and
a control unit for regulating the frequency of the light from the diodes.

11. The equipment of claim 10 where the light from the diodes is essentially monochromatic light having a wavelength substantially from 400 to 990 nanometers and a frequency substantially from 0.1 Hertz to 60 Kilo-Hertz.

12. The equipment of claim 11 where the frequency of the light from the diodes is maintained at essentially a first constant frequency for a predetermined period and then switched to a second frequency and maintained essentially constant at said second frequency for a predetermined period.

13. The equipment of claim 10 including a penile enlargement device.

14. The equipment of claim 13 where the penile enlargement device comprises a pumping apparatus.

15. Apparatus for enhancing the health of a penis comprising
penile illuminating equipment including a plurality of light emitting diodes positioned on a mounting structure in a predetermined manner to illuminate a penis with light emitted from the diodes substantially along the penis' entire length and circumference, and
a penile enlargement device for increasing the size of the penis detachably connected to the penile illuminating equipment.

16. The apparatus of claim 15 where the penile enlargement device comprises a pumping apparatus.

17. The apparatus of claim 16 including a control unit for regulating the frequency of the light from the diodes so that said light is essentially monochromatic light having a wavelength substantially from 400 to 990 nanometers and a frequency substantially from 0.1 Hertz to 60 Kilo-Hertz, said control unit automatically discontinuing the illumination after a predetermined time period and during said predetermined time period switching from one essentially constant frequency to another essentially constant frequency.

18. A method of enhancing the health of a penis comprising the step of illuminating the penis substantially along the penis' entire length and circumference with light from a plurality of light emitting diodes.

19. The method of claim 18 where said light is essentially monochromatic light having a wavelength substantially from 400 to 990 nanometers and a frequency substantially from 0.1 Hertz to 60 Kilo-Hertz.

20. The method of claim 18 where said light is modulated switching from one essentially constant frequency to another essentially constant frequency.

21. The method of claim 18 including subjecting the penis to a vacuum.

22. The method of claim 18 where the illumination is for a period substantially from 5 to 15 minutes for no more than 5 times per day.

23. The method of claim 18 where the illumination is from an array of diodes wherein substantially all the diodes emit light of substantially the same wavelength.
